(19) United States
(12) Patent Application Publication
(10) Pub. No.: US 2017/0113007 A1
(43) Pub. Date: Apr. 27, 2017

(54) THERMAL INSULATION SUCTION NOZZLE

(71) Applicant: SHENZHEN JIESHIBO TECHNOLOGY CO., LTD., Shenzhen (CN)

(72) Inventor: Jianyong Wu, Shenzhen (CN)

(21) Appl. No.: 14/988,751

(22) Filed: Jan. 6, 2016

(30) Foreign Application Priority Data
Oct. 27, 2015 (CN) 201520837688.9

(51) Int. Cl. A61M 15/00; A24F 47/00; A61M 15/06

(52) U.S. Cl. CPC A61M 15/0021; A61M 15/06; A24F 47/004; A61M 2205/3633

(57) ABSTRACT

A thermal insulation suction nozzle is disclosed, including a hollow inner tube, where an outer wall of the inner tube extends outwardly in a radial direction to form at least two partition plates, an adjusting ring is further sleeved on the inner tube in a rotatable manner, at least two partition spaces between the inner tube and the adjusting ring are partitioned by the partition plates, air inlet holes are disposed in a lower part of the adjusting ring, the number of the air inlet holes being the same as the number of the partition plates, and the adjusting ring is rotated outside the inner tube so as to enable the air inlet holes to communicate with or close the partition spaces. The thermal insulation suction nozzle has the advantages of preventing harms to the oral cavity caused by vapor and improving the degree of comfort for inhalation.

### CROSS-REFERENCE TO RELATED APPLICATIONS

The present application claims the benefit of Chinese Patent Application No. 201520837688.9 filed on Oct. 27, 2015; the contents of which are hereby incorporated by reference.

### BACKGROUND

Technical Field

The present application relates to an atomizer, and in particular, to a thermal insulation suction nozzle which can prevent harm to the oral cavity caused by vapor and improve the degree of comfort for inhalation.

Related Art

An atomizer is mainly used for inhalation of fragrance or medicine. Currently, the atomizer on market has a suction nozzle with a short air passage, and because vapor is not cooled, the temperature of the vapor entering the oral cavity of a human is too high, which may easily cause harm to the oral cavity, rendering uncomfortable feelings.

### SUMMARY

In order to solve the abovementioned problems, an objective of the present application is to provide a thermal insulation suction nozzle that can prevent harm to the oral cavity caused by vapor and improve the degree of comfort for inhalation.

The aforementioned objective is achieved in the present application through the following technical measures: a thermal insulation suction nozzle, including a hollow inner tube, where an outer wall of the inner tube extends outwardly in a radial direction to form at least two partition plates, an adjusting ring is further sleeved on the inner tube in a rotatable manner, at least two partition spaces between the inner tube and the adjusting ring are partitioned by the partition plates, air inlet holes are disposed in a lower part of the adjusting ring, the number of the air inlet holes being the same as the number of the partition plates, and the adjusting ring is rotated outside the inner tube so as to enable the air inlet holes to communicate with or close the partition spaces.

In a preferred example, an edge of each partition plate is provided with an arc-shaped structure that covers a corresponding air inlet hole in a matching manner.

In a preferred example, an outer tube is further disposed outside the inner tube, the outer wall of the inner tube is connected to an inner wall of the outer tube by the partition plates, middle holes that enable the air inlet holes to communicate with the partition spaces in a matching manner are disposed in the outer tube, and the adjusting ring is sleeved on an outer surface of the outer tube in a rotatable manner.

In a preferred example, the number of the partition plates is three.

In a preferred example, a sealing ring is disposed inside the adjusting ring.

In a preferred example, a connecting member for connecting an atomizer is disposed on an inner wall of the inner tube.

The thermal insulation suction nozzle of the present application is further provided with the adjusting ring outside the inner tube for taking in the high-temperature vapor, and a multiple-chamber space between the adjusting ring and the inner tube is used for taking in external air at the same time as taking in the high-temperature vapor. An air flow in an air inlet channel of the multiple chambers can be increased, reduced and closed by rotating and turning the adjusting ring, thereby cooling the high-temperature vapor entering the oral cavity of a human by using external cold air, so as to prevent harms to the oral cavity. Non-uniform inhalation is prevented by using the partition spaces, thereby enabling the temperature of the vapor inhaled into the mouth to be uniform, and improving the degree of comfort for inhalation.

### BRIEF DESCRIPTION OF THE DRAWINGS

FIG. 1 is a schematic structural diagram of an embodiment of the present application;

FIG. 2 is a front view of an embodiment of the present application;

FIG. 3 is a cross-sectional view along A-A in FIG. 2; and

FIG. 4 is a cross-sectional view along B-B in FIG. 2.

### DETAILED DESCRIPTION

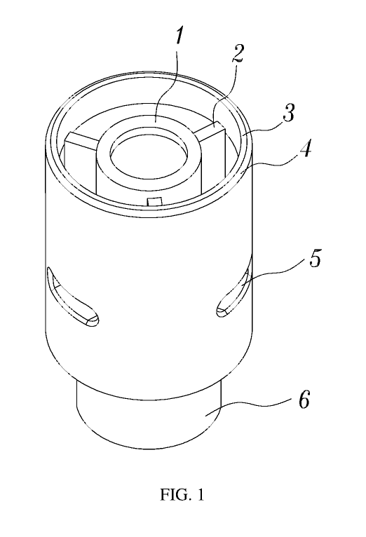

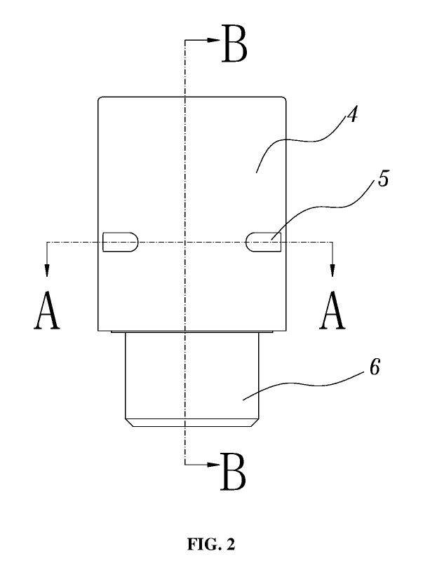

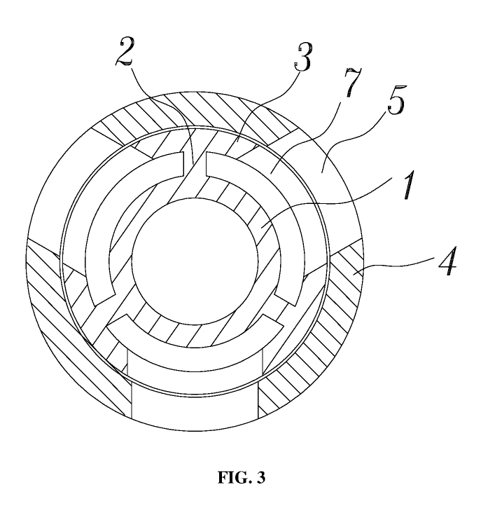

Referring to FIG. 1 to FIG. 4, a thermal insulation suction nozzle of this embodiment includes a hollow inner tube 1, an outer wall of the inner tube 1 extends outwardly in a radial direction to form at least two partition plates 2, an adjusting ring 4 is further sleeved on the inner tube 1 in a rotatable manner, multiple partition spaces between the inner tube and the adjusting ring 4 are partitioned by the partition plates 2, air inlet holes 5 are disposed in a lower part of the adjusting ring 4, the number of the air inlet holes 5 being the same as the number of the partition plates 2, and the adjusting ring 4 is rotated outside the inner tube so as to enable the air inlet holes 5 to communicate with or close the partition spaces.

The thermal insulation suction nozzle of this embodiment is further provided with the adjusting ring 4 outside the inner tube 1 for taking in the high-temperature vapor, and a multiple-chamber space between the adjusting ring 4 and the inner tube 1 is used for taking in external air at the same time as taking in the high-temperature vapor. An air flow in an air inlet channel of the multiple chambers can be increased, reduced and closed by rotating and turning the adjusting ring 4, thereby cooling the high-temperature vapor entering the oral cavity of a human by using external cold air, so as to prevent harms to the oral cavity. Non-uniform inhalation is prevented by using the partition spaces, thereby enabling the temperature of the vapor inhaled into the mouth to be uniform, and improving the degree of comfort for inhalation.

A thermal insulation suction nozzle according to another embodiment may specifically be that, on the basis of the previous technical solution, an edge of each partition plate is provided with an arc-shaped structure that covers a corresponding air inlet hole in a matching manner. Therefore, the air flow can be increased, reduced or closed by using the arc-shaped structure.

Referring to FIG. 1 to FIG. 4, the thermal insulation suction nozzle according to this embodiment may specifically be that, on the basis of the previous technical solution, an outer tube 3 is further disposed outside the inner tube 1, the outer wall of the inner tube 1 is connected to an inner wall of the outer tube 3 by three partition plates 2, middle holes 7 that enable the air inlet holes 5 to communicate with the partition spaces in a matching manner are disposed in the outer tube 3, and the adjusting ring 4 is sleeved on an outer surface of the outer tube 3 in a rotatable manner.

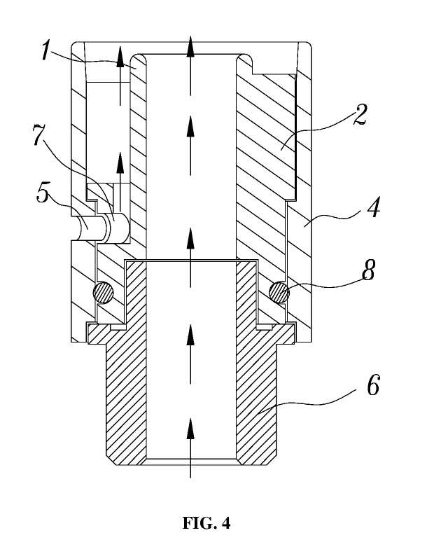

Referring to FIG. 4, the thermal insulation suction nozzle according to this embodiment may specifically be that, on the basis of the previous technical solution, a sealing ring 8 is disposed between the adjusting ring 4 and the outer surface of the outer tube 3, and the sealing ring 8 functions to prevent air leakage.

Referring to FIG. 1 to FIG. 4, the thermal insulation suction nozzle according to this embodiment may specifically be that, on the basis of the previous technical solution, a connecting member 6 for connecting an atomizer is disposed on an inner wall of the inner tube 1. After the connecting member 6 is tightly connected to the inner tube 1, a clearance fit can be constrained between the inner tube and the air inlet adjusting ring.

The above is to illustrate a thermal atomization device of the present application, so as to help understanding of the present application. However, the embodiments of the present application are not limited by the abovementioned embodiments. Any change, modification, replacement, combination, and simplification made within the principle of the present application should be equivalent replacing manners, which are all included in the protection scope of the present application.

What is claimed is:

1. A thermal insulation suction nozzle, comprising a hollow inner tube, wherein an outer wall of the inner tube extends outwardly in a radial direction to form at least two partition plates, an adjusting ring is further sleeved on the inner tube in a rotatable manner, at least two partition spaces between the inner tube and the adjusting ring are partitioned by the partition plates, air inlet holes are disposed in a lower part of the adjusting ring, the number of the air inlet holes being the same as the number of the partition plates, and the adjusting ring is rotated outside the inner tube so as to enable the air inlet holes to communicate with or close the partition spaces.

2. The thermal insulation suction nozzle according to claim 1, wherein an edge of each partition plate is provided with an arc-shaped structure that covers a corresponding air inlet hole in a matching manner.

3. The thermal insulation suction nozzle according to claim 1, wherein an outer tube is further disposed outside the inner tube, the outer wall of the inner tube is connected to an inner wall of the outer tube by the partition plates, middle holes that enable the air inlet holes to communicate with the partition spaces in a matching manner are disposed in the outer tube, and the adjusting ring is sleeved on an outer surface of the outer tube in a rotatable manner.

4. The thermal insulation suction nozzle according to claim 1, wherein the number of the partition plates is three.

5. The thermal insulation suction nozzle according to claim 2, wherein the number of the partition plates is three.

6. The thermal insulation suction nozzle according to claim 3, wherein the number of the partition plates is three.

7. The thermal insulation suction nozzle according to claim 1, wherein a sealing ring is disposed inside the adjusting ring.

8. The thermal insulation suction nozzle according to claim 2, wherein a sealing ring is disposed inside the adjusting ring.

9. The thermal insulation suction nozzle according to claim 3, wherein a sealing ring is disposed inside the adjusting ring.

10. The thermal insulation suction nozzle according to claim 1, wherein a connecting member for connecting an atomizer is disposed on an inner wall of the inner tube.

11. The thermal insulation suction nozzle according to claim 2, wherein a connecting member for connecting an atomizer is disposed on an inner wall of the inner tube.

12. The thermal insulation suction nozzle according to claim 3, wherein a connecting member for connecting an atomizer is disposed on an inner wall of the inner tube.
